(19) United States
(12) Patent Application Publication
(10) Pub. No.: US 2026/0198956 A1
(43) Pub. Date: Jul. 16, 2026

(54) SPHINCTEROTOME WITH DEFLECTING TIP

(71) Applicant: Boston Scientific Medical Device Limited, Galway (IE)

(72) Inventor: Deepak Kumar Sharma, Gurgaon (IN)

(73) Assignee: Boston Scientific Medical Device Limited, Galway (IE)

(21) Appl. No.: 19/447,162

(22) Filed: Jan. 13, 2026

Related U.S. Application Data
(60) Provisional application No. 63/745,033, filed on Jan. 14, 2025.

(51) Int. Cl. A61B 17/32; A61B 17/00; A61M 5/00; A61M 25/00; A61M 25/01

(52) U.S. Cl. CPC A61B 17/320016; A61M 5/007; A61M 25/0026; A61M 25/0147; A61B 2017/00238; A61B 2017/00314; A61B 2017/00327; A61B 2017/00818; A61B 2017/32006; A61M 2025/015

(57) ABSTRACT

A steerable medical device may include a proximal elongate shaft having a first plurality of lumens and a distal elongate shaft having a second plurality of lumens, wherein the second plurality is less than the first plurality. An articulation assembly may couple the proximal and distal elongate shafts. The articulation assembly may include a segmented sleeve coupled to the proximal elongate shaft and an articulating connector pivotably coupled to the segmented sleeve and coupled to the distal elongate shaft. At least one deflection member may extend through a dedicated lumen of the proximal elongate shaft and may be couple to the articulating connector. Selective actuation of the deflection member may cause lateral deflection of the distal elongate shaft relative to the proximal elongate shaft.

### CROSS-REFERENCE TO RELATED APPLICATIONS

This application claims the benefit of U.S. Provisional Patent Application Ser. No. 63/745,033 filed on Jan. 14, 2025, the disclosure of which is incorporated herein by reference.

### FIELD

The disclosure pertains to medical devices, and methods for manufacturing and use of these medical devices. More particularly, the present disclosure pertains to medical devices for accessing a body lumen along a biliary and/or pancreatic tract.

### BACKGROUND

Endoscopic retrograde cholangiopancreatography (ERCP) is a medical procedure that combines upper gastrointestinal endoscopy and x-ray imaging to diagnose and treat conditions affecting the bile ducts and the pancreatic ducts. The procedure utilizes specialized endoscopic equipment, such as a duodenoscope, which is inserted through the patient's mouth and guided through the esophagus and stomach to the duodenum. During ERCP procedures, physicians navigate the scope while maintaining control of its proximal end for various functions including irrigation, visualization, insufflation, and device management. The procedure enables the exchange of specialized devices, such as ERCP cannulas, through a biopsy port for both diagnostic and therapeutic interventions. These cannulas facilitate access to the pancreaticobiliary system, allowing for guidewire placement, contrast dye injection, and various therapeutic maneuvers under x-ray guidance. There is an ongoing need to provide alternative cannulation devices for accessing ducts, such as, but not limited to, the common bile duct and the pancreatic duct as well as alternative methods for manufacturing and using medical devices.

### SUMMARY

This disclosure provides design, material, manufacturing method, and use alternatives for medical devices and medical systems.

In an example, a sphincterotome device may include a proximal elongate shaft having a first plurality of lumens, a distal elongate shaft having a second plurality of lumens, wherein the second plurality may be less than the first plurality, an articulation assembly coupling the proximal and distal elongate shafts that may include a segmented sleeve coupled to the proximal elongate shaft and an articulating connector pivotably coupled to the segmented sleeve and coupled to the distal elongate shaft, and at least one deflection member extending through a lumen of the proximal elongate shaft and coupled to the articulating connector, wherein selective actuation of the at least one deflection member may cause lateral deflection of the distal elongate shaft relative to the proximal elongate shaft.

Alternatively or additionally to any of the examples above, in another example, the first plurality of lumens may comprise five lumens and the second plurality of lumens may comprise three lumens.

Alternatively or additionally to any of the examples above, in another example, the device may further comprise an intermediate elongate shaft extending through the articulation assembly and connecting corresponding lumens between the proximal and distal elongate shafts through the articulation assembly.

Alternatively or additionally to any of the examples above, in another example, the segmented sleeve may comprise first and second slots extending from a proximal end thereof and first and second apertures positioned between the slots.

Alternatively or additionally to any of the examples above, in another example, the articulating connector may comprise first and second linkage members pivotably coupled to the segmented sleeve via pins extending through the first and second apertures.

Alternatively or additionally to any of the examples above, in another example, the at least one deflection member may comprise first and second deflection members positioned on opposing sides of the articulating connector.

Alternatively or additionally to any of the examples above, in another example, the first plurality of lumens may comprise a guidewire lumen, a fluid lumen, and a plurality of control element lumens.

Alternatively or additionally to any of the examples above, in another example, the second plurality of lumens may comprise a guidewire lumen, a fluid lumen, and a wire lumen.

Alternatively or additionally to any of the examples above, in another example, the articulation assembly may enable lateral deflection while maintaining lumen continuity between the proximal and distal elongate shafts.

Alternatively or additionally to any of the examples above, in another example, the handle assembly may comprise a first actuator for controlling a cutting wire and a second actuator for controlling lateral deflection of the distal elongate shaft.

Alternatively or additionally to any of the examples above, in another example, the at least one deflection member may extend through a lumen in the proximal elongate shaft separate from working lumens for guidewire passage and fluid delivery.

Alternatively or additionally to any of the examples above, in another example, the articulation assembly may enable lateral deflection of the distal elongate shaft of 45 degrees or more relative to a longitudinal axis of the segmented sleeve.

Alternatively or additionally to any of the examples above, in another example, actuation of the deflection members may enable lateral deflection of the distal elongate shaft beyond 90 degrees or more relative to a longitudinal axis of the segmented sleeve.

Alternatively or additionally to any of the examples above, in another example, the articulation assembly may enable controlled lateral deflection of the distal elongate shaft through at least 120 degrees of movement.

Alternatively or additionally to any of the examples above, in another example, the device may further comprise a wire filament extending through the proximal elongate shaft and at least a portion of the distal elongate shaft, the wire filament may be actuatable to curve a distal end region of the distal elongate shaft.

In an example, a steerable medical device may include a proximal elongate shaft having a first plurality of lumens, a distal elongate shaft having a second plurality of lumens, wherein the second plurality may be less than the first plurality, an articulation assembly coupling the proximal and distal elongate shafts that may include a segmented sleeve coupled to the proximal elongate shaft, an articulating connector pivotably coupled to the segmented sleeve and coupled to the distal elongate shaft, and at least one deflection member extending through a dedicated lumen of the proximal elongate shaft and coupled to the articulating connector, wherein selective actuation of the at least one deflection member may cause lateral deflection of the distal elongate shaft relative to the proximal elongate shaft.

Alternatively or additionally to any of the examples above, in another example, the device may further comprise an intermediate elongate shaft configured to connect corresponding lumens between the proximal and distal elongate shafts through the articulation assembly.

Alternatively or additionally to any of the examples above, in another example, the segmented sleeve may comprise first and second slots extending from a proximal end thereof and first and second apertures positioned between the slots.

Alternatively or additionally to any of the examples above, in another example, the articulating connector may comprise first and second linkage members pivotably coupled to the segmented sleeve via pins extending through the first and second apertures.

Alternatively or additionally to any of the examples above, in another example, the at least one deflection member may comprise first and second deflection members positioned on opposing sides of the articulating connector.

Alternatively or additionally to any of the examples above, in another example, the first plurality of lumens may comprise five lumens and the second plurality of lumens may comprise three lumens.

Alternatively or additionally to any of the examples above, in another example, the articulation assembly may enable lateral deflection of the distal elongate shaft beyond 90 degrees relative to a longitudinal axis of the proximal elongate shaft.

In an example, a medical device may include an elongate shaft having a proximal elongate shaft and a distal elongate shaft, the proximal elongate shaft may comprise a first lumen configuration, the distal elongate shaft may comprise a second lumen configuration different from the first lumen configuration, a hinge assembly coupling the proximal and distal sections that may include a clevis structure mounted on pivot pins, and a first control element and a second control element each coupled to the clevis structure for controlling lateral deflection, wherein actuation of the deflection members may cause controlled lateral movement of the distal elongate shaft relative to the proximal elongate shaft.

Alternatively or additionally to any of the examples above, in another example, the first lumen configuration may comprise a guidewire lumen, a contrast lumen, a steering wire lumen, and two or more control element lumens.

Alternatively or additionally to any of the examples above, in another example, the second lumen configuration may comprise a guidewire lumen, a contrast lumen, and a wire lumen.

Alternatively or additionally to any of the examples above, in another example, the device may further comprise an intermediate elongate shaft defining a plurality of lumens, the plurality of lumens extending between corresponding lumens of the proximal and distal elongate shafts through the hinge assembly.

Alternatively or additionally to any of the examples above, in another example, the first and second control elements may comprise deflection wires extending through dedicated lumens in the proximal section.

Alternatively or additionally to any of the examples above, in another example, the hinge assembly and the intermediate elongate shaft may enable lateral deflection of the distal elongate shaft while maintaining lumen continuity between the proximal and distal elongate shafts.

Alternatively or additionally to any of the examples above, in another example, actuation of the control elements may enable lateral deflection of the distal section beyond 90° relative to a longitudinal axis of the proximal section.

In an example, a medical system may include a handle assembly, a proximal elongate shaft extending from the handle assembly, an articulation assembly at a distal portion of the proximal elongate shaft, a plurality of deflection members extending from the handle assembly through the proximal elongate shaft to the articulation assembly, a distal elongate shaft extending distally from the articulation assembly, wherein the handle assembly may comprise actuation mechanisms configured to selectively tension the deflection members to control lateral movement of the distal elongate shaft.

Alternatively or additionally to any of the examples above, in another example, the handle assembly may comprise a first actuator for controlling a cutting wire and a second actuator for controlling lateral deflection.

Alternatively or additionally to any of the examples above, in another example, the articulation assembly may comprise a pivotable connector.

Alternatively or additionally to any of the examples above, in another example, the proximal elongate shaft may comprise a first number of lumens and the distal elongate shaft may comprise a second number of lumens less than the first number.

Alternatively or additionally to any of the examples above, in another example, the deflection members may extend through dedicated lumens in the proximal elongate shaft.

Alternatively or additionally to any of the examples above, in another example, the articulation assembly may enable controlled lateral deflection through at least 120° of movement.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various configurations and together with the description serve to explain the principles of the present disclosure.

FIG. 1 is a schematic view of an illustrative system for accessing an opening of a body lumen of a subject;

FIG. 2 is a schematic view of the system depicted in FIG. 1, taken from circle-2;

FIG. 3 is a schematic view of an illustrative system for accessing a body lumen;

FIG. 4 is a perspective view of a distal end region of the illustrative accessory device;

FIG. 5A is a cross-sectional view of the proximal elongate shaft taken at line 5A-5A of FIG. 4;

FIG. 5B is a cross-sectional view of the distal elongate shaft taken at line 5B-5B of FIG. 4;

FIG. 6 is an exploded perspective view of the articulation assembly;

FIG. 7 is a schematic cross-sectional view of the articulation assembly coupled with the distal and proximal elongate shafts;

FIG. 8 is a perspective view of the accessory device at the articulation assembly with the articulating connector and distal elongate shaft removed;

FIG. 9 is an enlarged perspective view of the articulation assembly showing the deflection capabilities;

FIG. 10 is a side view of the handle assembly; and

FIG. 11 is a partial cross-sectional view of a portion of the handle assembly.

While the disclosure is amenable to various modifications and alternative forms, specifics thereof have been shown by way of example in the drawings and will be described in detail. It should be understood, however, that the intention is not to limit the invention to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

Although configurations of the present disclosure may be described with specific reference to medical devices and systems (e.g., endoscopic devices, accessory tools, and/or guidewires inserted through a duodenoscope, near or through a papilla, or the like) for selective access to, aligning with, and/or cannulation of the common bile duct (CBD) or pancreatic duct (PD) during an Endoscopic Retrograde Cholangiopancreatography (ERCP) procedure, it should be appreciated that such medical devices and systems may be used in a variety of medical procedures which require navigating one or more accessory tools through ductal, luminal, vascular, or body lumen anatomies, including, for example, interventional radiology procedures, balloon angioplasty/angiography procedures, thrombolysis procedures, urological or gynecological procedures, and the like. The disclosed medical devices and systems may be inserted via different access points and approaches, e.g., percutaneously, endoscopically, laparoscopically, or some combination thereof.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features, and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed configuration(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “a configuration”, “some configurations”, “other configurations”, etc., indicate that the configuration(s) described may include a particular feature, structure, or characteristic, but every configuration may not necessarily include the particular feature, structure, or characteristic. Moreover, such phrases are not necessarily referring to the same configuration. Further, when a particular feature, structure, or characteristic is described in connection with a configuration, it would be within the knowledge of one skilled in the art to effect the particular feature, structure, or characteristic in connection with other configurations, whether or not explicitly described, unless clearly stated to the contrary. That is, the various individual elements described below, even if not explicitly shown in a particular combination, are nevertheless contemplated as being combinable or arrangeable with each other to form other additional configurations or to complement and/or enrich the described configuration(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some configurations, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

As used in this disclosure, the terms “upper” and “lower” are relative terms used to differentiate between opposite directions, not the overall orientation of the device. One of ordinary skill will recognize that a device could operate, for example, with the “uppermost” component at the bottom and the “lowermost” component at the top (i.e., “upside-down” when comparing the device operation to the terms), or in any other absolute orientation relative to the user and/or gravity.

The detailed description is intended to illustrate but not limit the disclosure. Those skilled in the art will recognize that the various elements described may be arranged in various combinations and configurations without departing from the scope of the disclosure. The detailed description illustrates example configurations of the disclosure.

In some instances, the devices and methods that are disclosed herein may be useful for diagnostic or therapeutic procedures in the biliary and/or pancreatic tracts, along with being useful for other purposes. Access to the pancreaticobiliary system, as facilitated by the devices disclosed herein, may be required to diagnose and/or treat a variety of conditions, including but not limited to tumors, gallstones, infections, sclerosis, and pseudo cysts. The devices disclosed herein may also be useful for navigation in other parts of the body such as the cardiovascular system and so forth.

Endoscopic retrograde cholangiopancreatography (ERCP) may be used to diagnose and treat conditions of the common bile duct, including, for example, gallstones, inflammatory strictures, leaks (e.g., from trauma, surgery, etc.), and cancer. In an ERCP procedure, a physician may view, through an endoscope, the inside of the stomach and/or the duodenum. Often, dyes may be injected (e.g., via a lumen of a sphincterotome or other device) into the ducts in the biliary tree and pancreas so that the area can be seen using X-rays. These procedures may necessitate gaining and keeping access to the papilla of Vater, the common bile duct, and/or the pancreatic duct, which may be technically challenging, may require extensive training and practice to gain proficiency, and may require one or more expensive tools in order to perform.

During an ERCP procedure, a number of steps are typically performed while the patient is often sedated and/or anaesthetized. For example, an endoscope or duodenoscope may be inserted through the mouth, down the esophagus, into the stomach, through the pylorus into the duodenum, to a position at or near the papilla of Vater (also referred to as the ampulla of Vater), which is the opening of the common bile duct and the pancreatic duct. Due to the shape of the papilla, and the angle at which the common bile and pancreatic ducts meet the wall of the duodenum, the distal end of the endoscope or duodenoscope is generally placed just past the papilla. Due to this positioning beyond the papilla, the endoscopes or duodenoscopes typically used in these procedures are usually side-viewing devices. The side-viewing feature provides imaging along the lateral aspect of the tip rather than from the end of the endoscope. Such orientation may allow a clinician to obtain an image of the medial wall of the duodenum, where the papilla of Vater is located, even though the distal tip of the viewing device is beyond the opening. Once the papilla or a target area in the duodenum is visually located, an accessory medical device, such as sphincterotome, cannula, or other accessory medical device, may be extended out a side opening or window of the endoscope or duodenoscope for facilitating access through the papilla and into a desired one of the common bile duct and the pancreatic duct.

Although not required, a fluid (e.g., a compressible fluid, such as compressible gas, air, nitrogen, carbon dioxide, etc., and/or other suitable fluid) may be delivered via a catheter (e.g., as inserted through a subject as discussed above or otherwise inserted), a sphincterotome, a cannula, and/or other elongated tubular device extending through an inserted endoscope, and/or other suitable elongate medical device to an opening (e.g., the papilla of Vater) of a body lumen of a subject. Applying the fluid to the opening may facilitate identifying and/or accessing the body lumen by at least partially dilating the opening with the fluid prior to and/or during delivery of a catheter, sphincterotome, guidewire, etc. to a target anatomical structure or tissue (e.g., a duct or other suitable structure or tissue) at or distal of the opening of the body lumen. Some example techniques and systems for cannulating an opening of a body lumen using fluid are described in U.S. Patent Application Publication No. 2019/0380565, which was filed on Jun. 13, 2019, and titled DEVICES, SYSTEMS AND METHOD FOR ACCESSING A BODY LUMEN, which is hereby incorporated by reference for any and all purposes.

Alternatively or in addition to using fluid to open the opening of the body lumen, the opening may be manually opened by engaging a distal end or tip of an accessory medical device with the papilla. Further, the accessory medical device may be manipulated and/or the endoscope through which the accessory medical device extends may be manipulated to direct the accessory medical device through the papilla and into a desired one of the common bile duct and the pancreatic duct. Some example techniques and systems for manipulating a distal tip of a medical device, such as a guidewire, to access a desired body lumen are described in U.S. Patent Application Publication No. 2015/0073391, which was filed on Sep. 11, 2014, and titled MEDICAL DEVICE WITH A MOVABLE TIP, which is hereby incorporated by reference for any and all purposes.

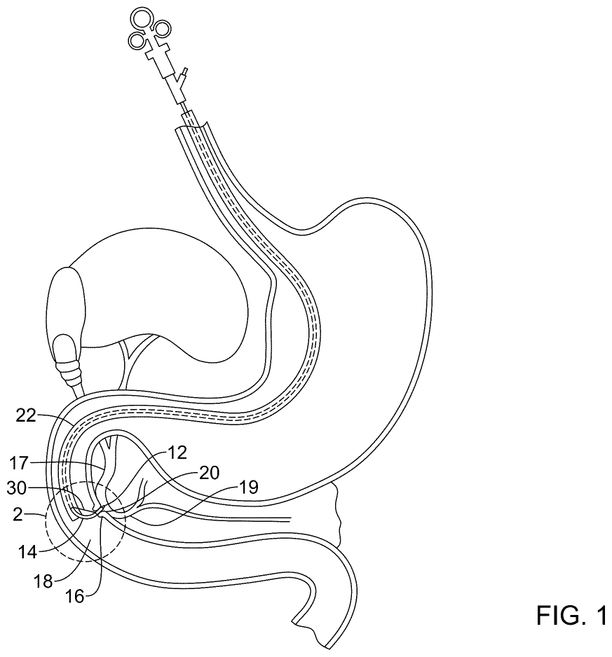

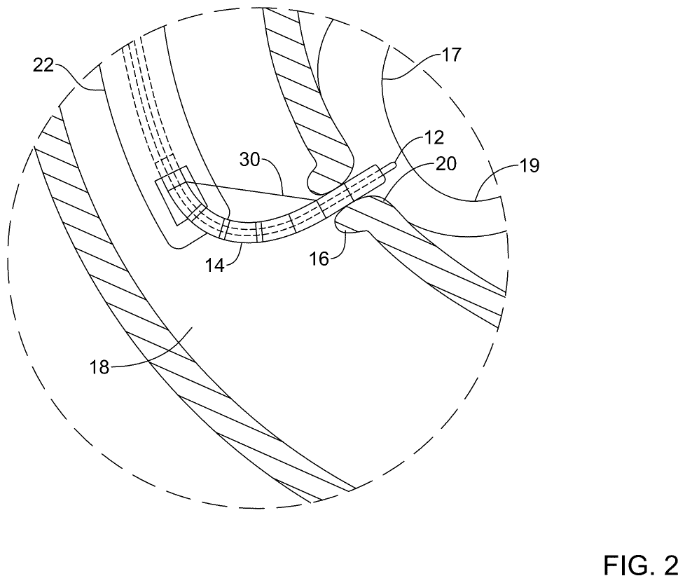

Turning to the Figures, FIGS. 1 and 2 depict a selective cannulation during an ERCP procedure, which includes a guidewire 12 and/or an accessory medical device 14 (e.g., an endoscopic accessory device, such as a cannula, a sphincterotome, and/or other suitable accessory medical device) being passed towards, against, and/or through a body lumen such as the major papilla 16 (e.g., ampullary entry) near the descending duodenum 18 to access the Sphincter of Oddi Complex 20. During the cannulation procedure, a distal portion of a medical device 22 (e.g., an endoscope, duodenoscope, guide catheter, etc.) may be positioned within the descending duodenum 18. The guidewire 12 and the accessory medical device 14 may be advanced through a working channel (e.g., a lumen) of the medical device 22 towards the major papilla 16. Additionally, the guidewire 12 and/or the accessory medical device 14 may be advanced against and/or through the major papilla 16 to one of the common bile duct 17 and the pancreatic duct 19.

Accessing the papilla 16, the common bile duct 17, and/or the pancreatic duct 19 may be difficult because the openings are small compared to a size (e.g., a diameter or other size measurement) of many accessory medical devices, the openings may be closely located, the openings may be completely collapsed/closed, the openings may extend into the descending duodenum 18 at an angle that may be difficult to visualize and/or access, and/or it may be difficult to control a position of a distal tip of the accessory medical device 14. Thus, a medical professional may be required to manipulate the accessory medical device 14 and guidewire 12 by manually rotating the medical device 22, pulling on a wire filament 30 to adjust the accessory medical device 14 in a single direction, and/or use an elevator and/or other suitable ramped surface within the distal end of the medical device 22 in an attempt to align or orient the accessory medical device 14 and/or the guidewire 12 with respect to the medical device 22 and the openings of the papilla 16, the common bile duct 17, and/or the pancreatic duct 19. Difficult cannulation procedures in which the medical professional fails to access the body lumen within a certain time limit or after a certain number of unsuccessful attempts may lead to significant post-procedure complications, such as post-ERCP pancreatitis (PEP).

A medical professional accessing a body lumen (e.g., a duct, a papilla, a common bile duct, a pancreatic duct, or the like) by manipulating an accessory medical device against or into the opening of the body lumen, as discussed above, may subject the walls of the opening of the body lumen to compressive forces. Compression of a body lumen opening or the body lumen itself can cause buckling in what may be described as the “accordion effect”. As such, it is desirable to have control over a distal tip of the accessory medical device 14 such that a user may precisely access an opening of the papilla 16 and once through the papilla 16, direct the accessory medical device 14 and/or guidewire 12 to a desired one of the common bile duct 17 and the pancreatic duct 19.

In some configurations, approaches to papilla cannulation may include devices that utilize rotating pull wires to rotate the distal tip. While these devices achieve some degree of tip orientation through passive steerability by causing twist at the distal bowing section, they have limitations in providing precise control. For example, an entirety of the shaft may be rotated which creates lag between actuation of the wires and movement of the distal tip. Further, release of the steering wires may cause the shaft to rotate quickly in an uncontrolled manner in the opposing direction. Disclosed herein are cannulation devices with active tip deflection capabilities.

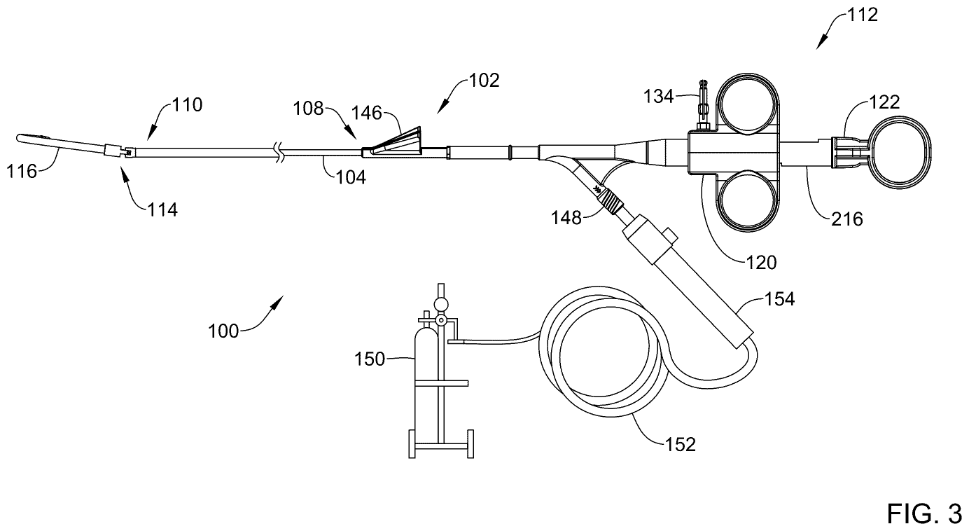

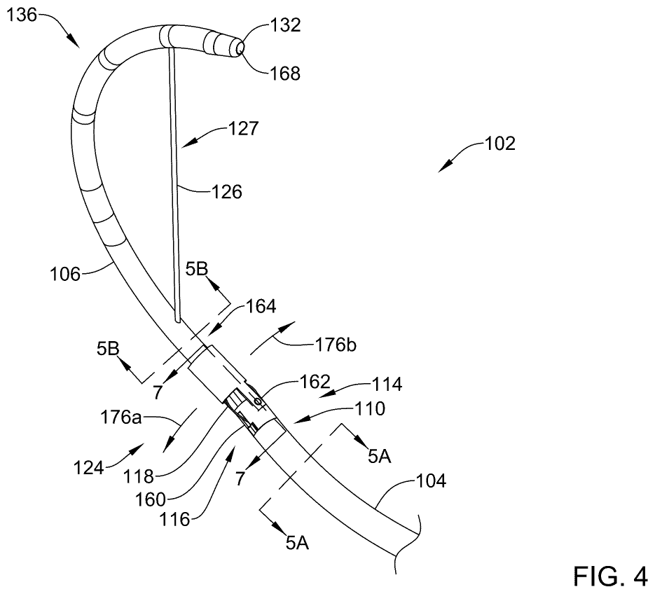

FIG. 3 schematically depicts an illustrative system 100 for accessing a body lumen. In some cases, the system 100 may include, among other components, an accessory medical device 102, similar in form and function to the accessory medical device 14, with a proximal flexible elongate shaft or tube 104 and a distal flexible elongate shaft or tube 106. The proximal and/or distal elongate shafts 104, 106 may be extruded to define a plurality of lumens extending therethrough. In some examples, the accessory medical device 102 may be a sphincterotome that may be used with another medical device, such as, but not limited to, the medical device 22 described herein. While the accessory device 102 is described as a sphincterotome, the device 102 may be a cannula or other accessory device, as desired. The proximal elongate shaft 104 may extend from a proximal end region 108 (disposed within or coupled to a handle assembly 112) to a distal end region 110. As will be described in more detail herein, the distal end region 110 of the proximal elongate shaft 104 may be coupled to the distal elongate shaft 106 via a hinge assembly or an articulation assembly 114 and an intermediate member 116 having at least one flexible elongate tube 118 (see, for example, FIG. 4).

The handle assembly 112 may be any suitable type of handle. In one example, the handle assembly 112 may have a first portion or actuator 120 configured to receive fingers (e.g., receive two fingers) of a user using the accessory medical device 102 to hold the accessory medical device 102 and a second portion or actuator 122 configured to receive a thumb or other finger of the user. In some cases, the first portion 120 of the handle assembly 112 may be an actuating portion of the handle assembly 112 and a user may adjust the first portion 120 relative to a third portion 216 to control positioning of a distal end of the accessory medical device 102 in a first direction. In some cases, the second portion 122 of the handle assembly 112 may also be an actuating portion of the handle assembly 112 and a user may adjust the second portion 122 relative to the third portion 216 to control positioning of a distal end of the accessory medical device 102 in a second direction. As schematically depicted in FIG. 3, the first portion 120 of the handle assembly 112 may have two finger holes configured to receive two fingers from the user and the second portion 122 may have a thumb or finger hole configured to receive a thumb or finger from the user, but other configurations of the first and second portions 120, 122 of the handle assembly 112 are contemplated. Although not required, the handle assembly 112, as illustrated, may be held and/or operated with a single hand of a user.

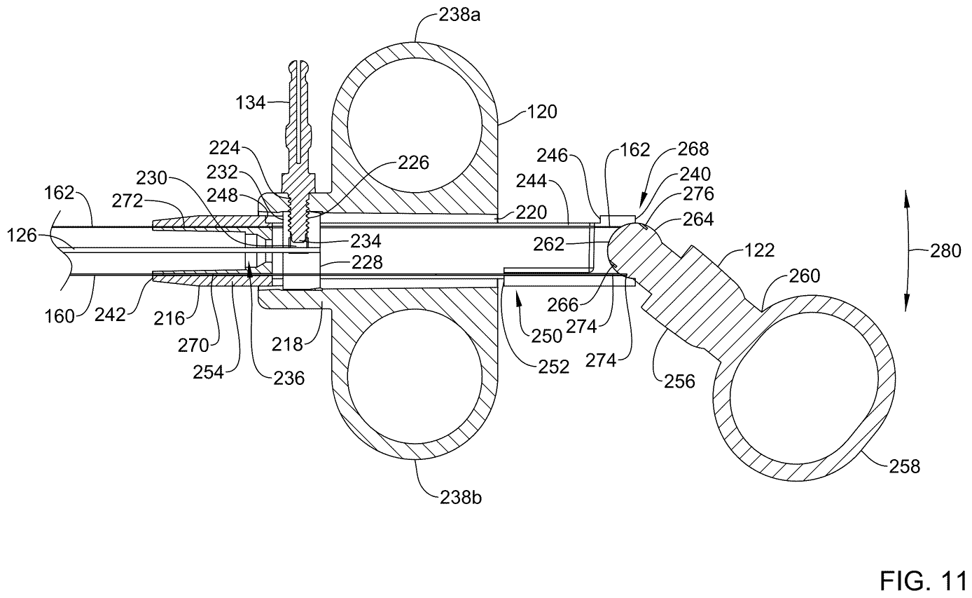

Referring additionally to FIG. 4 which is a perspective view of a distal end region 124 of the illustrative accessory device 102, a wire filament 126 may extend along the proximal elongate shaft 104 and at least partially along the distal elongate shaft 106. The wire filament 126 may be actuated to control or steer the distal end region or the distal elongate shaft 106 of the accessory device 102. In some cases, the wire filament 126 may be a steering wire. The wire filament 126 may have a proximal end region 236 (see, for example, FIG. 11) connected to the first portion 120 of the handle assembly 112, and a proximal portion extending within the proximal elongate shaft 104 and at least partially within the distal elongate shaft 106. The wire filament 126 also may have a distal end (not explicitly shown) connected to the distal end region of the distal elongate shaft 106. In some configurations, the distal end of the wire filament 126 may be secured to the distal elongate shaft 106 within a lumen thereof (e.g., lumen 130). The wire filament 126 may be a 304V stainless steel spring temper wire. However, other materials may be used, as desired.

The first portion 120 of the handle assembly 112 may be configured to be adjusted or manipulated to control a position of a distal end region of the distal elongate shaft 106. In some cases, the first portion 120 may be configured to adjust axially or longitudinally to adjust a position of the distal elongate shaft 106. Alternatively, or additionally, in some cases, the first portion 120 may be configured to adjust radially or rotationally to adjust a position of the distal elongate shaft 106.

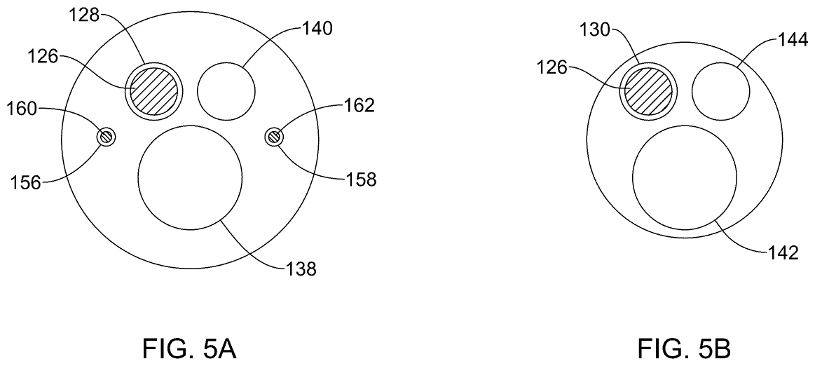

The wire filament 126 may be received within a lumen 128 (see, for example, FIG. 5A) of the proximal elongate shaft 104 at least along a proximal portion of the filament 126 and may be configured to slide within the lumen 128. The wire filament 126 may be at least partially received within a lumen 130 (see, for example, FIG. 5B) of the distal elongate shaft 106. In some cases, the first portion 120 of the handle assembly 112 may be coupled to the wire filament 126 and manipulated to slide at least a portion of the wire filament 126 along the proximal elongate shaft 104 and/or distal elongate shaft 106 (e.g., within the lumens 128, 130 thereof). In one example, the first portion 120 of the handle assembly 112 may be longitudinally adjusted relative to the second portion 122 of the handle assembly 112 to apply and remove tension to and/or from the wire filament 126.

Sliding or adjusting the wire filament 126 may result in the distal elongate shaft 106 of moving responsively to the sliding of the wire filament 126. As such, the first portion 120 of the handle assembly 112 may be manipulated to control or adjust a position of the distal elongate shaft 106, for example, to direct the distal end 132 toward the opening of a body lumen. For example, proximal actuation of the wire filament 126 may move the distal elongate shaft 106 from a generally linear configuration to a generally curved configuration. In some cases, natural tension in or acting on the distal elongate shaft 106 and/or the wire filament 126 may result in the wire filament 126 returning to a relaxed position or state once a tension applied to the wire filament 126 via the handle assembly 112 is released.

In some cases, the wire filament 126 may be utilized to electrically cut or remove tissue of the subject (e.g., to cut and cauterize tissue) and may be considered a cautery wire. The wire filament 126 also may have a distal portion 127 (e.g., a cutting portion) extending external to the distal elongate shaft 106. The handle assembly 112 or other suitable portion of the medical device 22 may include an electrical connection at a connection member 134 for an energy source (e.g., a radiofrequency energy source, or the like) to energize the wire filament 126 and facilitate cutting tissue. However, this is not required. Alternatively, or additionally the connection member 134 may be releasably secured to the wire filament 126.

The distal end region 136 of the distal elongate shaft 106 may be flexible and configured to adjust in response to steering with or adjustment of a control configuration at a proximal end of the accessory medical device 102. To facilitate precisely controlling a distal end of the accessory medical device 102 while the device 102 is within a subject, the distal end region 136 may be configured to be adjustable or steerable in two or more directions through control at the proximal end of the accessory medical device 102 such that distal elongate shaft 106 may be guided to a desired body lumen. The distal end region 136 may be a steerable distal tip.

The distal end region 136 or distal elongate shaft 106 may be formed from any suitable material configured to bend, deflect, or otherwise move in response to forces acting thereon. In one example, the distal tip may be formed from a polyether block amide (PEBA, such as, for example, PEBAX®), but this is not required, and other suitable materials are contemplated.

A positioning of the distal end 132 may be controlled or adjusted in any suitable manner. In one example, the wire filament 126 may be actuated to control a position of the distal end 132. As will be described in more detail herein, a distal end of the wire filament 126 may be coupled to the distal elongate shaft 106 and/or a proximal end of the wire filament 126 may be coupled to the first portion 120 of the handle assembly 112 in any suitable manner. In one example, the wire filament 126 may be coupled to the distal elongate shaft 106 and/or the first portion 120 of the handle assembly 112 using adhesion, for example, through ultraviolet (UV) curing techniques, crimping techniques, mechanical fixation techniques, and/or other suitable connecting or coupling techniques.

Referring additionally to FIG. 5A, which is a cross-sectional view of the illustrative proximal elongate shaft 104, taken at line 5A-5A of FIG. 4, in addition to the one or more lumens 128 in which the wire filament 126 for controlling a position of the distal end 132 and/or distal elongate shaft 106 extends, the proximal elongate shaft 104 may include one or more other lumens extending from at least the proximal end region 108 of the proximal elongate shaft 104 to the distal end region 110 of the proximal elongate shaft 104. For example, the proximal elongate shaft 104 may include a guidewire lumen 138 and a fluid lumen 140. The guidewire lumen 138 may be configured to receive a guidewire (not explicitly shown) therethrough. The fluid lumen 140 may be configured to receive a contrast fluid, a pressurized fluid, saline, or the like.

In some cases, one or more of the lumens 128, 138, 140 may each have a distal opening (not explicitly shown) at the distal end region 110 of the proximal elongate shaft 104. The one or more lumens 128, 138, 140 may be configured to be fluidly coupled with one or more lumens 130, 142, 144 of the distal elongate shaft 106, as will be described in more detail herein. In some cases, a guidewire or other suitable medical devices may extend through the guidewire lumen 138 of the proximal elongate shaft 104 and proximally out of a first aperture port 146. The aperture port 146 may be located at the distal end of the handle assembly 112 and/or at one or more other suitable locations. In some cases, the aperture port 146 and the proximal elongate shaft 104 may be configured so that the guidewire and/or other suitable medical devices may be stripped through the side of the proximal elongate shaft 104 for a rapid removal or exchange of devices, but this is not required.

In some cases, the fluid lumen 140 of the proximal elongate shaft 104 may have a fluid connection at a proximal end for a fluid source (e.g., a pressurized fluid source 150 such as a CO₂ tank, a contrast fluid source such as a syringe, etc.) The lumen 140 may be configured to apply a fluid from the fluid source through an opening at the distal end region 110 of the proximal elongate shaft 104 to the distal elongate shaft 106 and finally to a body lumen of the subject.

Although FIG. 3 depicts the fluid source 150 coupled to a fluid port 148 of the accessory medical device 102 via a fluid tube 152 and a flow regulator 154, the fluid source 150 and/or other suitable fluid sources (e.g., syringes, etc.) may be coupled to the fluid port 148 in other suitable manners. In one example, a syringe with contrast fluid may be coupled to the fluid port 148 via a luer lock connection and/or other suitable connection. Contrast agent may be delivered into the body lumen and may be used to temporarily improve imaging of the inside of the body lumen by, for example, x-ray, computed tomography (CT), or magnetic resonance (MR) imaging, ultrasound, and the like. Alternatively, separate lumens and separate fluid ports may be provided for each of a pressurized fluid and a contrast fluid. In some cases, the fluid source 150 may be omitted.

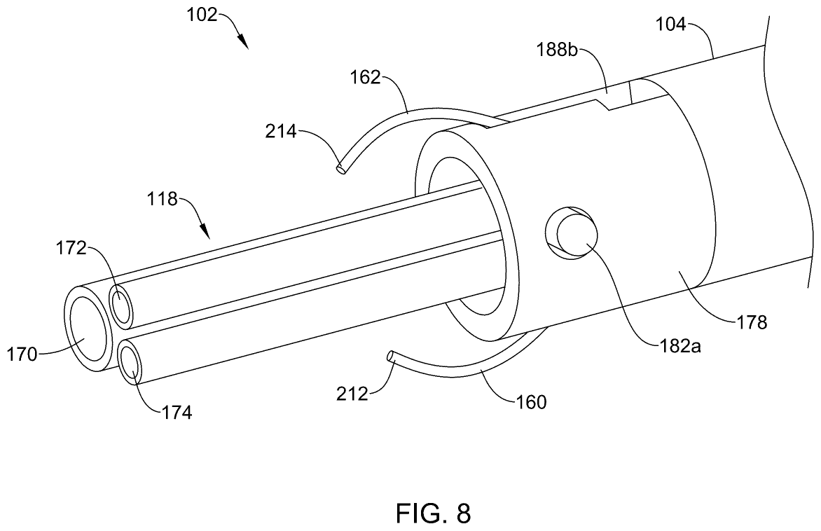

The proximal elongate shaft 104 may further include a first deflection member lumen 156 and a second deflection member lumen 158. The first and second deflection member lumens 156, 158 may be configured to each receive a control element or deflection member 160, 162 therein. The first and second deflection member lumens 156, 158 may be fluidly and mechanically isolated from the steering wire lumen 128, the guidewire lumen 138, and the fluid lumen 140. The deflection members 160, 162 may extend distally from the second portion 122 through the proximal elongate shaft 104. The deflection members 160, 162 may be actuated via the second portion 122 to control or steer the distal elongate shaft 106 in a direction different from the steering provided through actuation of the wire filament 126. A distal end 212, 214 (see, for example FIG. 8) of each of the deflection members 160, 162 may extend distally beyond a distal end of the proximal elongate shaft 104 and are coupled with the articulation assembly 114. A proximal end 274, 276 (see, for example FIG. 11) of each of the deflection members 160, 162 may be connected to the second portion 122 of the handle assembly 112. The deflection members 160, 162 may be a 304V stainless steel spring temper wire. However, other materials may be used, as desired.

Referring additionally to FIG. 5B, which is a cross-sectional view of the illustrative distal elongate shaft 106, taken at line 5B-5B of FIG. 4, in addition to the one or more lumens 130 in which the wire filament 126 for controlling a position of the distal end 132 and/or distal elongate shaft 106 extends, the distal elongate shaft 106 may include one or more other lumens extending from at least the proximal end region 164 of the distal elongate shaft 106 to the distal end region 136 of the distal elongate shaft 106. For example, the distal elongate shaft 106 may include a guidewire lumen 142 and a fluid lumen 144. The guidewire lumen 142 may be configured to receive a guidewire (not explicitly shown) therethrough. The fluid lumen 144 may be configured to receive a contrast fluid, a pressurized fluid, saline, or the like.

In some cases, one or more of the lumens 130, 142, 144 may each have a proximal opening (not explicitly shown) at the proximal end region 164 of the distal elongate shaft 106. The guidewire lumen 142 and the fluid lumen 144 may extend distally from the proximal openings to merge to a common a distal opening 168. While the guidewire lumen 142 and the fluid lumen 144 are shown and described as having a common distal opening 168, the guidewire lumen 142 and the fluid lumen 144 may have separate distal openings as desired. Further, the distal openings may be positioned proximal to the distal end 132 of the distal elongate shaft 106, if so desired. The wire filament or steering lumen 130 may or may not include a distal opening, as desired. The one or more lumens 130, 142, 144 may be configured to be fluidly coupled with one or more lumens 128, 138, 140 of the proximal elongate shaft 104, as will be described in more detail herein. As the deflection members 160, 162 are connected to the articulation assembly 114 proximal to the distal elongate shaft 106, the distal elongate shaft 106 may be free from lumens to accommodate the deflection members 160, 162. Thus, the distal elongate shaft 106 may have fewer lumens that the proximal elongate shaft 104. In the illustrated configuration, the proximal elongate shaft 104 may include five lumens and the distal elongate shaft 106 may include three lumens. However, the proximal elongate shaft 104 may include more than five or less than five lumens. Similarly, the distal elongate shaft 106 may include more than three or less than three lumens.

Returning to FIG. 4, the distal elongate shaft 106 may be pivotably coupled to the proximal elongate shaft 104 via the hinge assembly or an articulation assembly 114 and an intermediate member 116 having a flexible elongate tube 118. The proximal and distal elongate shafts 104, 106, may be formed as separate components that are subsequently coupled together. Further, the articulation assembly 114 and the intermediate member 116 may be formed as separate components from the proximal and distal elongate shafts 104, 106, and may be subsequently coupled therewith.

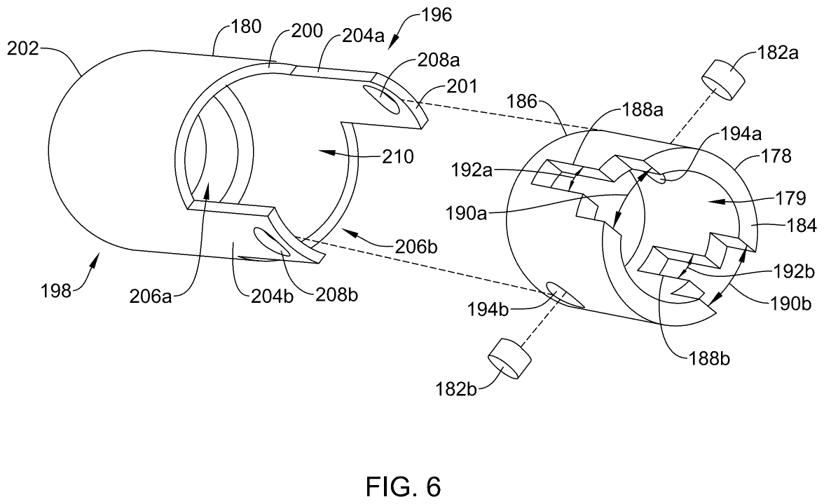

The articulation assembly 114 may be configured to be actuated to pivot or deflect the distal elongate shaft 106 in a direction different from the deflection generated by actuation of the wire filament 126. For example, actuation of the wire filament 126 may curl or bend the distal elongate shaft 106 proximally while actuation of the articulation assembly 114 may move the distal elongate shaft 106 laterally, as shown at arrows 176a, 176b. FIG. 6 is an exploded perspective view of the articulation assembly 114. The articulation assembly 114 may include a segmented sleeve or collar 178 and a clevis structure or articulating connector 180. The articulating connector 180 may be pivotably or rotatably coupled to the segmented sleeve 178 via a first pin 182a and a second pin 182b.

The segmented sleeve 178 may be a generally tubular member extending from a proximal end 184 to a distal end 186 and defining a lumen 179 extending from the proximal end 184 to the distal end 186. The segmented sleeve 178 may include a first slot 188a and a second slot 188b extending through a thickness of the sidewall of the segmented sleeve 178 (e.g., from an outer surface to an inner or luminal surface thereof). The slots 188a, 188b may be positioned on opposing sides of the segmented sleeve 178 or may be spaced approximately 180° from one another. The slots 188a, 188b extend distally from the proximal end 184 of the segmented sleeve 178. The slots 188a, 188b may extend less than an entire length of the segmented sleeve 178. For example, the slots 188a, 188b may terminate or end proximal to the distal end 186 of the segmented sleeve 178. The slots 188a, 188b may have a first width (or arc length) 190a, 190b adjacent the proximal end 184. The slots 188a, 188b may transition to a second, smaller, width (or arc length) 192a, 192b in an abrupt or stepwise manner at an intermediate location. In other configurations, the slots 188a, 188b may transition from the first width 190a, 190b to the second width 192a, 192b in a tapered, sloped, or gradual manner. In yet other configurations, the slots 188a, 188b may have a uniform width along a length thereof. The second width 192a, 192b may be sized to allow a first deflection member 160 and a second deflection member 162 to extend radially from the slots 188a, 188b.

The segmented sleeve 178 may further include a first aperture 194a and a second aperture 194b extending through a thickness of the sidewall of the segmented sleeve 178. The first and second apertures 194a, 194b may have a generally circular cross-sectional shape. However, the first and second apertures 194a, 194b may take other cross-sectional shapes, as desired. The first and second apertures 194a, 194b may be sized and shaped to receive the first and second pins 182a, 182b, respectively. In some examples, the first and second pins 182a, 182b may be fixedly or releasably secured within the first and second apertures 194a, 194b. However, this is not required. The first and second pins 182a, 182b may be configured to extend radially beyond an outer surface of the segmented sleeve 178 such that the articulating connector 180 may be rotatably coupled to the segmented sleeve 178 via the first and second pins 182a, 182b. The apertures 194a, 194b may be positioned on opposing sides of the segmented sleeve 178 or may be spaced approximately 180° from one another. In some configurations, the apertures 194a, 194b may each be positioned circumferentially between the slots 188a, 188b such that the first aperture 194a is spaced approximately 90° from each of the first and second slots 188a, 188b and the second aperture 194b is spaced approximately 90° from each of the first and second slots 188a, 188b. Said differently, the first and second slots 188a, 188b and the first and second apertures 194a, 194b may be generally uniformly spaced about a circumference of the segmented sleeve 178 in an alternating configuration. The apertures 194a, 194b may be positioned at similar axial locations to one another anywhere along a length of the segmented sleeve 178.

The proximal end region of the segmented sleeve 178 may be configured to be secured to the distal end region 110 of the proximal elongate shaft 104. In some examples, the proximal end region of the segmented sleeve 178 may be disposed over and surround an outer surface of the distal end of the proximal elongate shaft 104. The proximal end region of the segmented sleeve 178 may be secured to the distal end of the proximal elongate shaft 104 using adhesion, for example, through ultraviolet (UV) curing techniques, crimping techniques, mechanical fixation techniques, and/or other suitable connecting or coupling techniques.

The articulating connector 180 may extend from a proximal end 201 to a distal end 202 and may include a proximal end region 196 and a distal end region 198. A lumen or opening 210 may extend from the proximal end 201 to the distal end 202. The distal end region 198 of the articulating connector 180 may be generally tubular and may extend distally from an intermediate location 200 to the distal end 202 of the articulating connector 180. The distal end region 198 may have a first inner diameter adjacent to the intermediate location 200 and a second inner diameter adjacent to the distal end 202 of the articulating connector 180. In some examples, the second inner diameter may be smaller than the first inner diameter. However, this is not required. In other configurations, the second inner diameter may be greater than the first inner diameter. In yet other configurations, the inner diameter of the distal end region 198 may be substantially constant. The second inner diameter may be similar in size to an outer diameter of a proximal end region 164 of the distal elongate shaft 106. The proximal end region 164 of the distal elongate shaft 106 may be coupled to the distal end region 198 of the articulating connector 180 using adhesion, for example, through ultraviolet (UV) curing techniques, crimping techniques, mechanical fixation techniques, and/or other suitable connecting or coupling techniques.

The proximal end region 196 may include a first arm or linkage member 204a and a second arm or linkage member 204b. The linkage members 204a, 204b may each extend proximally from the intermediate location 200 to the proximal end 201 of the articulating connector 180. Each of the first and second linkage members 204a, 204b may have an arc length that is less than half the circumference of the distal end region 198 of the articulating connector 180 to define a first gap or open space 206a between the first and second linkage members 204a, 204b and a second gap or open space 206b between the first and second linkage members 204a, 204b. The gaps 206a, 206b may be sized and shaped to allow the deflection members 160, 162 to extend radially beyond an outer diameter of the articulating connector 180 and to allow for proximal and/or distal movement of the deflection members 160, 162. The linkage members 204a, 204b may be positioned on opposing sides of the articulating connector 180 or spaced approximately 180° from one another.

The linkage members 204a, 204b may each include an aperture 208a, 208b extending through a thickness thereof. In some configurations, the apertures 208a, 208b may be positioned adjacent to the proximal end 201 of the articulating connector 180. However, this is not required. The apertures 208a, 208b may be positioned at any axial location along the linkage members 204a, 204b, as desired. The apertures 208a, 208b may be positioned at similar axial locations to one another. The apertures 208a, 208b may have a generally circular cross-sectional shape. However, this is not required. The apertures 208a, 208b may be sized and shaped to receive a respective pin 182a, 182b therethrough. It is contemplated that that aperture 208a, 208b may be free from adhesives, glue, or other mechanical coupling mechanisms that would prevent rotation of the articulating connector 180 relative to the pins 182a, 182b. For example, when assembled the articulating connector 180 is pivotably coupled to the segmented sleeve 178 via the pins 182a, 182b to form a hinge joint. Said differently, the pins 182a, 182b extend through the apertures 208a, 208b of the articulating connector 180 and the apertures 194a, 194b of the segmented sleeve 178 to movably couple the articulating connector 180 to the segmented sleeve 178.

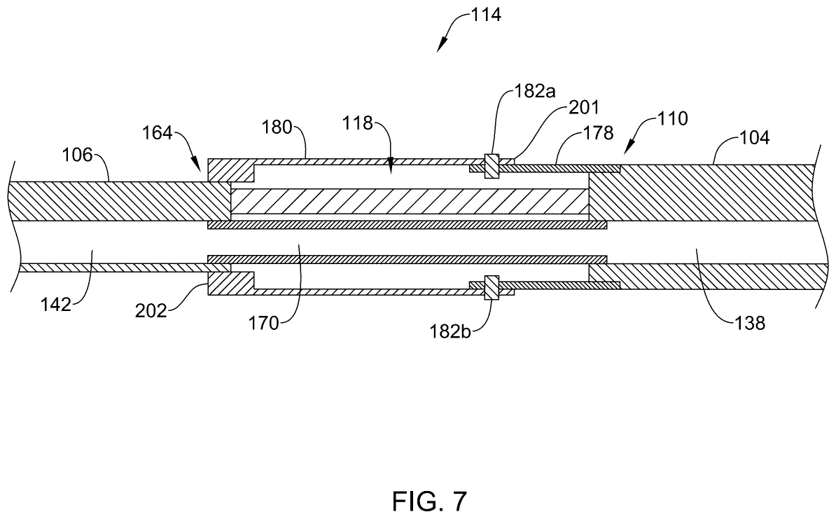

FIG. 7 is a schematic cross-sectional view of the illustrative articulation assembly 114 coupled with the distal elongate shaft 106 and the proximal elongate shaft 104, taken at line 7-7 of FIG. 4. As described with respect to FIG. 6, the proximal end region of the segmented sleeve 178 may be coupled with a distal end or distal end region 110 of the proximal elongate shaft 104 and the distal end region 198 of the articulating connector 180 may be coupled with a proximal end or proximal end region 164 of the distal elongate shaft 106. The intermediate elongate shaft 118 may extend between and be coupled with each of the distal elongate shaft 106 and the proximal elongate shaft 104. The intermediate elongate shaft 118 may be flexible such that as the articulation assembly 114 is actuated, the intermediate elongate shaft 118 bends with the movement of the articulation assembly 114. The intermediate elongate shaft 118 may include one or more lumens extending between and fluidly coupling one or more lumens of the proximal elongate shaft 104 and the distal elongate shaft 106. For example, referring additionally to FIG. 8 which is a perspective view of the accessory device 102 at the articulation assembly 114 with the articulating connector 180 and the distal elongate shaft 106 removed, the intermediate elongate shaft 118 may include a first lumen 170 configured to extend between and fluidly couple the guidewire lumen 138 of the proximal elongate shaft 104 and the guidewire lumen 142 of the distal elongate shaft 106, a second lumen 172 configured to extend between and fluidly couple the steering wire lumen 128 of the proximal elongate shaft 104 and the steering wire lumen 130 of the distal elongate shaft 106, and a third lumen 174 configured to extend between and fluidly couple the fluid lumen 140 of the proximal elongate shaft 104 and the fluid lumen 144 of the distal elongate shaft 106. The intermediate elongate shaft 118 may be coupled with the proximal elongate shaft 104 and the distal elongate shaft 106 to provide a fluid tight connection between the proximal elongate shaft 104 and the distal elongate shaft 106. The intermediate elongate shaft 118 may be formed from a plurality of separate tubular members. In other configurations, the intermediate elongate shaft 118 may be a single monolithic multi-lumen member.

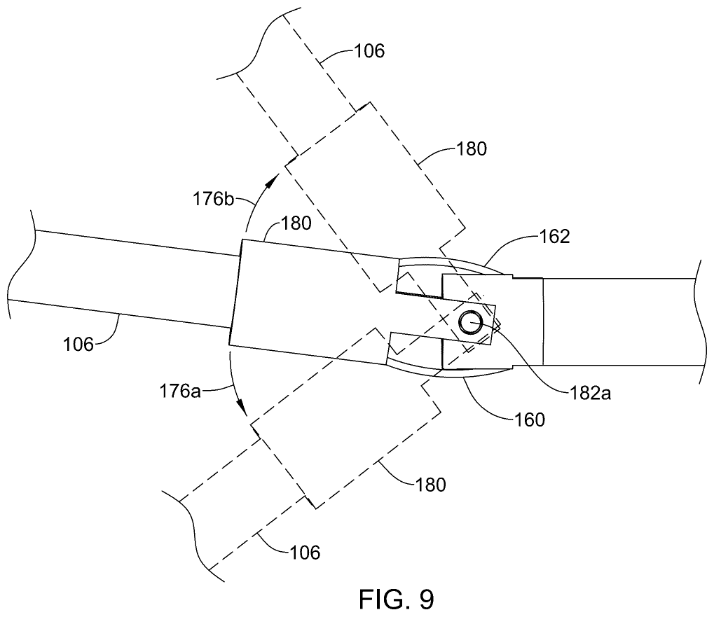

FIG. 9 is an enlarged perspective view of the articulation assembly 114. The distal end 212 (see, for example, FIG. 8) of the first deflection member 160 may be fixedly secured to the distal end region 198 of the articulating connector 180 at a luminal surface of the articulating connector 180. For example, the distal end 212 of the first deflection member 160 may be coupled to the articulating connector 180 adjacent to the intermediate location 200 at a first fixation location using techniques such as adhesion, for example, through ultraviolet (UV) curing techniques, crimping techniques, mechanical fixation techniques, and/or other suitable connecting or coupling techniques. The first fixation location of the first deflection member 160 on the articulating connector 180 may be circumferentially aligned with the first slot 188a. Similarly, the distal end 214 (see, for example, FIG. 8) of the second deflection member 162 may be fixedly secured to the distal end region 198 of the articulating connector 180 at a luminal surface of the articulating connector 180. For example, the distal end 214 of the second deflection member 162 may be coupled to the articulating connector 180 adjacent to the intermediate location 200 at a second fixation location using techniques such as adhesion, for example, through ultraviolet (UV) curing techniques, crimping techniques, mechanical fixation techniques, and/or other suitable connecting or coupling techniques. The second fixation location of the second deflection member 162 on the articulating connector 180 may be circumferentially aligned with the second slot 188b.

The articulating connector 180 may provide controlled deflection between proximal elongate shaft 104 and the distal elongate shaft 106 of the accessory device 102. For example, the articulating connector 180 may be pivotally mounted between the proximal elongate shaft 104 and the distal elongate shaft 106 via the laterally extending pins 182a, 182b. Positioning the pins 182a, 182b on opposing sides allow the articulating connector 180 to pivot, hinge, deflect, or otherwise move sideways or laterally relative to the longitudinal axis of the accessory device 102. The articulation assembly 114 may enable controlled lateral deflection of the distal elongate shaft 106 (relative to the longitudinal axis of the segmented sleeve 178 and/or the longitudinal axis of the proximal elongate shaft 104) through at least 90° or more, at least 120° or more, at least 150° or more, at least 180° or more of movement. Said differently, the distal elongate shaft 106 may be deflected plus or minus 45° or more from the longitudinal axis of the segmented sleeve 178, plus or minus 60°, or more, from the longitudinal axis of the segmented sleeve 178, plus or minus 75° or more from the longitudinal axis of the segmented sleeve 178, plus or minus 90° or more from the longitudinal axis of the segmented sleeve 178, or the like. It is contemplated that the length of the linkage members 204a, 204b may be selected to achieve the desired range of deflection. For example, increasing the length of the linkage members 204a, 204b may increase the range of motion.

The deflection members 160, 162 extend through dedicated wire lumens 156, 158 in the proximal elongate shaft 104 that are separate from the guidewire lumen 135 and the fluid lumen 140. This may allow for independent control of the deflection members 160, 162 while maintaining functionality of the guidewire lumens 138, 142 and the fluid lumens 140, 144 during deflection. By applying varying tensions to individual deflection members 160, 162, the articulating connector 180 can be selectively deflected to angle the distal elongate shaft 106 relative to the proximal elongate shaft 104. The pivoting action may occur at the pins 182a, 182b which form a pivot axis perpendicular to the longitudinal axis of the proximal elongate shaft 104. When tension is applied through at least one deflection member 160, 162, the articulating connector 180 rotates about this pivot axis, causing controlled lateral deflection of the distal elongate shaft 106. For example, a proximal force may be applied to the first deflection member 160 to move the distal elongate shaft 106 in a first direction 176a (towards the first deflection member 160). Proximal retraction of the first deflection member 160 may be countered by distal advancement of the second deflection member 162. A proximal force may be applied to the second deflection member 162 to move the distal elongate shaft 106 in a second direction 176b (towards the second deflection member 162). Proximal retraction of the second deflection member 162 may be countered by distal advancement of the first deflection member 160. The pins 182a, 182b are securely mounted to maintain proper pivoting action while preventing unwanted movement in other directions. The intermediate elongate shaft 118 may bend as the articulating connector 180 is deflected to maintain a fluid tight connection between the lumens 128, 138, 140 of the proximal elongate shaft 104 and the lumens 130, 142, 144 of the distal elongate shaft 106. This may maintain continuity of the steering wire lumens 128, 130, the guidewire lumens 138, 142, and fluid lumens 140, 144 while allowing the required deflection movement. The intermediate elongate shaft 118 may be configured to accommodate the range of motion of the articulating connector 180 without compromising lumen integrity.

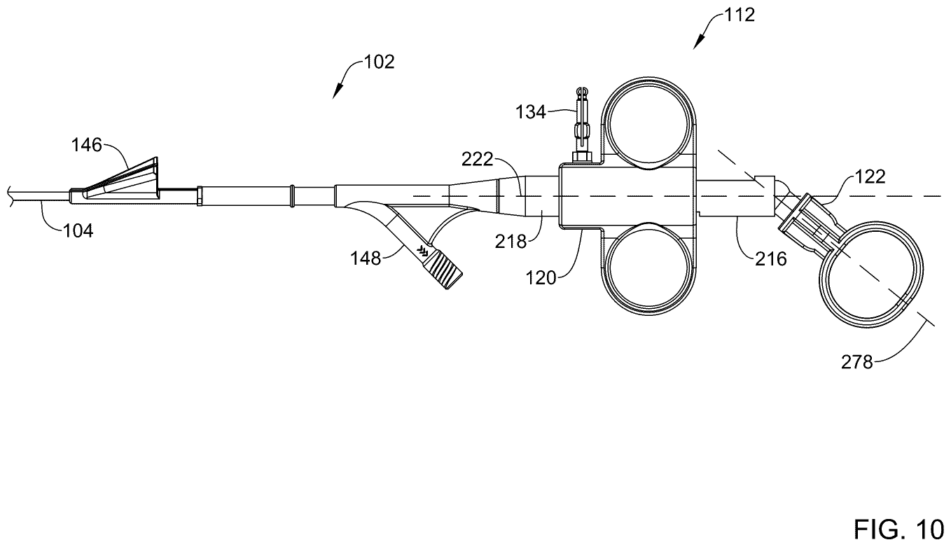

The wire filament 126, the first deflection member 160, and the second deflection member 162 may each be actuated at their respective proximal ends to selectively deflect the distal elongate shaft 106 in different directions or planes. FIG. 10 is a side view of the illustrative handle assembly 112 that may be used to actuate the wire filament 126 and the first and second deflection members 160, 162 to deflect, bend, or curve the distal elongate shaft 106. FIG. 11 is a cross-sectional view of a portion of the illustrative handle assembly 112. In FIGS. 10 and 11, the first portion 120 of the handle assembly 112 is in a deflected configuration in which the distal end region 136 of the distal elongate shaft 106 is curved in a first direction and the second portion 122 of the handle assembly 112 is actuated to laterally deflect the distal elongate shaft 106 such that the distal elongate shaft 106 extends at a non-parallel angle to the longitudinal axis of the proximal elongate shaft 104 and/or at a non-parallel angle to the longitudinal axis of the segmented sleeve 178.

As described herein, the handle assembly 112 may include an actuatable first portion 120, an actuatable second portion 122, and a third portion 216. Generally, the first portion 120 of the handle assembly 112 may be axially displaceable relative to the third portion 216 of the handle assembly 112. For example, the first portion 120 may be proximally retracted relative to the third portion 216 to deflect or bend the distal end region 136 of the distal elongate shaft 106 in a first direction and distally advanced relative to the third portion 216 to deflect or bend the distal end region 136 of the distal elongate shaft 106 in a second direction opposition the first direction. For example, proximal retraction of the first portion 120 may move the distal end 132 in a direction towards the exit location of the wire filament 126 and distal advancement of the first portion 120 may move the distal end 132 in a direction away from the exit location of the wire filament 126. Said differently, proximal retraction of the first portion 120 may bend the distal end region 136 such that the wire filament 126 is within an interior of the curved portion and distal advancement of the first portion 120 may bend the distal end region 136 such that the wire filament 126 extends along an exterior or outer region of the curved portion.

The first portion 120 may include a generally tubular main body portion 218 defining a lumen 220 extending from proximal end to a distal end of the tubular main body portion 218. The lumen 220 may extend generally coaxially with a proximal end region of the proximal elongate shaft 104. The first portion 120 may further include an opening or aperture 224 extending through a sidewall of the main body portion and extending generally orthogonal to the longitudinal axis 222 of the third portion 216. The aperture 224 may be configured to receive the connection member 134 therethrough. The connection member 134 may include a threaded region 226 configured to threadably engage a mating threaded region of a mounting member 228. The mounting member 228 may be slidably disposed within the lumen 220 of the main body portion 218. The mounting member 228 may include a first aperture 230 extending from a proximal end to a distal end of the mounting member 228. The first aperture 230 may be configured to receive a proximal end region 236 of the wire filament 126 therethrough. The mounting member 228 may further include a second aperture 232 extending generally orthogonal to the first aperture 230. The threaded region of the mounting member 228 may be formed in the luminal wall of the second aperture 232. The second aperture 232 may be connected to the first aperture 230 to allow a radially inward portion 234 of the connection member 134 to contact the wire filament 126. For example, once the wire filament 126 is secured to the distal elongate shaft 106, the proximal end region 236 of the wire filament 126 may be inserted into the first aperture 230 of the mounting member 228. The connection member 134 may then be rotated to move radially inwards to secure the proximal end region 236 of the wire filament 126 between the connection member 134 and the mounting member 228. As the proximal end region 236 of the wire filament 126 is secured to the first portion 120 of the handle assembly 112 via the mounting member 228 and the connection member 134, movement of the first portion 120 may be translated to the wire filament 126.

The first portion 120 of the handle assembly 112 may include a first ring member 238a and a second ring member 238b extending laterally from the main body portion 218 and each configured to receive a finger. The clinician may pull proximally on the ring members 238a, 238b to actuate the distal end region 136 of the distal elongate shaft 106 in a first direction or push distally on the ring members 238a, 238b to actuation the distal end region 136 of the distal elongate shaft 106 in a second direction, opposite the first direction.

The third portion 216 of the handle assembly 112 may include a generally tubular body portion 254 extending from a proximal end 240 to a distal end 242. The body portion 254 may include a slot 244 extending through a thickness or across a diameter thereof. The slot 244 may extend from a proximal end 246 to a distal end 248. The mounting member 228 of the first portion 120 of the handle assembly 112 may be axially displaceable along the slot 244. In some configurations, the slot 244 may not extend across an entirety of the diameter of the body portion 254 for an entire length of the slot 244. For example, the slot 244 may extend through less than an entirety of the diameter of the body portion 254 along a proximal portion 250 of the slot 244 to define a mechanical stop 252. Said differently, slot 244 may be configured to create a mechanical stop 252 along a region thereof to limit movement of the first portion 120 of the handle assembly 112. For example, as the first portion 120 is proximally retracted a proximal end of the mounting member 228 may contact the mechanical stop 252 to prevent further proximal movement of the first portion 120. It is contemplated that the length of the slot 244 which does not extend across the entire diameter of the body portion 254 may be selected based on the desired proximal movement of the first portion 120 (and the desired amount of bend at the distal elongate shaft 106).

It is further contemplated that a mechanical stop may be provided to limit distal movement of the first portion 120 of the handle assembly 112. In some cases, the mechanical stop may be the distal end 248 of the slot 244. For example, the distal end of the mounting member 228 may contact the distal end 248 of the slot 244 to limit distal movement of the first portion 120 of the handle assembly 112. In other configurations, the slot 244 may extend through less than an entirety of the diameter of the body portion 254 (similar to portion 250) to define a mechanical stop.

The first portion 120 of the handle assembly 112 may be assembled with the third portion 216 such that the body portion 254 of the third portion 216 is disposed within the lumen 220 of the main body portion 218 of the first portion 120. The first portion 120 may be axially displaceable along an outer surface of the third portion 216. The mounting member 228, the connection member 134 and the wire filament 126 may extend into the slot 244 of the third portion.

The second portion 122 of the handle assembly 112 may include a generally solid body portion 256 extending from a proximal end 260 to a distal end 262. A ring member 258 may extend proximally from the proximal end 260 of the body portion 256. The ring member 258 may be configured to receive a finger to move or adjust the second portion 122 of the handle assembly 112 to actuate the first deflection member 160 and/or the second deflection member 162. The body portion 256 may include a generally spherical connection member 264 adjacent the distal end 262 thereof. The connection member 264 may be configured to be received within a mating opening 266 formed in the proximal end 240 of the third portion 216 of the handle assembly 112 to form a ball joint 268. The second portion 122 of the handle assembly 112 may be rotated or pivoted relative to the third portion 216 to actuation the first deflection member 160 and/or the second deflection member 162. In some cases, the connection member 264 and/or the mating opening 266 may include features to limit movement of the second portion 122 to a single plane. For example, portions of the connection member 264 and the mating opening may be flattened to prevent rotation in directions other than the desired plane.

The first deflection member 160 and the second deflection member 162 may extend through lumens 270, 272 formed in the sidewall of the tubular body 254 of the third portion 216. The proximal ends 274, 276 of the first deflection member 160 and the second deflection member 162 may be coupled or secured to the connection member 264 or other portions of the body portion 256. The proximal ends 274, 276 are secured to an outer surface of the second portion such that as the second portion 122 is actuated in the desired plane (shown at arrow 280 in FIG. 11), tension is applied to one of the first deflection member 160 or the second deflection member 162 and tension is released from the other of the first deflection member 160 or the second deflection member 162. The first deflection member 160 and/or the second deflection member 162 may be secured to the second portion 122 using adhesion, for example, through ultraviolet (UV) curing techniques, crimping techniques, mechanical fixation techniques, and/or other suitable connecting or coupling techniques.

In the illustrated example, the second portion 122 has been deflected in a first direction such that a longitudinal axis 278 of the body portion 256 extends at a non-parallel angle to the longitudinal axis 222 of the third portion 216. When the second portion 122 is deflected in the first direction, tension is applied to the second deflection member 162 and released or relieved from the first deflection member 160. This may allow the articulating connector 180 to rotate about the pivot axis and deflect the distal elongate shaft towards the second deflection member 162 (as shown at arrow 176b in FIG. 9). The second portion 122 may be deflected in a second direction, opposite the first direction, to apply tension to the first deflection member 160 and release or relieve tension from the second deflection member 162. This may allow the articulating connector 180 to rotate about the pivot axis and deflect the distal elongate shaft towards the first deflection member 160 (as shown at arrow 176a in FIG. 9). When the longitudinal axis 278 of the body portion 256 extends generally parallel to or co-axial with the longitudinal axis 222 of the third portion 216, a longitudinal axis of the articulating connector 180 may extend parallel to or co-axial with the longitudinal axis of the segmented sleeve 178.

It is contemplated that the first portion 120 and the second portion 122 of the handle assembly 112 may be actuated independently to move the distal end region 136 to the desired orientation. However, it should be understood that both the first portion 120 and second portion 122 can be actuated substantially simultaneously or sequentially to both bend or curve the distal end region 136 as well as laterally deflect the distal elongate shaft 106.

The fluid port 148 may be coupled with the third portion 216 of the handle assembly 112 using known techniques, such as, but not limited to, press-fits, friction fits, over-molding, heat-shrinking, or the like. The proximal end region 108 of the proximal elongate shaft 104 may be coupled with the fluid port 148 using known techniques, such as, but not limited to, press-fits, friction fits, over-molding, heat-shrinking, mechanical coupling members such as pins, or the like. Further, the aperture port 146 may be coupled with the proximal elongate shaft 104 using known techniques, such as, but not limited to, over-molding, heat-shrinking, or the like. The proximal elongate shaft 104 may include side ports adjacent to the aperture port 146 to allow a guidewire to enter the guidewire lumen 138 from the aperture port 146.

The materials that can be used for the various components of the systems presently disclosed may include those commonly associated with medical devices. For simplicity purposes, the following discussion makes reference to system 100 and accessory device 102 referenced above. However, this is not intended to limit the devices and methods described herein, as the discussion may be applied to other similar devices and/or components of devices disclosed herein.

The system 100, accessory device 102, and/or components thereof may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include, but are not limited to, polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some configurations the sheath can be blended with a liquid crystal polymer (LCP). For example, the mixture can contain up to about 6 percent LCP.

In some configurations, a degree of Magnetic Resonance Imaging (MRI) compatibility is imparted into the system 100 and/or accessory device 102. For example, the system 100 and/or accessory device 102 or portions thereof may be made of a material that does not substantially distort the image and create substantial artifacts (i.e., gaps in the image). Certain ferromagnetic materials, for example, may not be suitable because they may create artifacts in an MRI image. The system 100 and/or accessory device 102 or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

As alluded to above, the tubular and/or elongated components of the system 100 and/or accessory device 102 may include one or more tubular members that may have slots formed therein. Various configurations of arrangements and configurations of slots are contemplated. For example, in some configurations, at least some, if not all of the slots are disposed at the same or a similar angle with respect to the longitudinal axis of the tubular and/or elongated components of the system 100 and/or accessory device 102. The slots can be disposed at an angle that is perpendicular, or substantially perpendicular, and/or can be characterized as being disposed in a plane that is normal to the longitudinal axis of the tubular and/or elongated components of the system 100 and/or accessory device 102. However, in other configurations, the slots can be disposed at an angle that is not perpendicular, and/or can be characterized as being disposed in a plane that is not normal to the longitudinal axis of the tubular components of the system 100 and/or accessory device 102. Additionally, a group of one or more the slots may be disposed at different angles relative to another group of one or more the slots. The distribution and/or configuration of the slots can also include, to the extent applicable, any of those disclosed in U.S. Pat. No. 7,914,467, the entire disclosure of which is herein incorporated by reference. Some example configurations of appropriate micromachining methods and other cutting methods, and structures for tubular members including slots and medical devices including tubular members are disclosed in U.S. Pat. Publication Nos. 2003/0069522 and 2004/0181174-A2; and U.S. Pat. Nos. 6,766,720; and 6,579,246, the entire disclosures of which are herein incorporated by reference. Some example configurations of etching processes are described in U.S. Pat. No. 5,106,455, the entire disclosure of which is herein incorporated by reference. It should be noted that the methods for manufacturing the system 100 and/or accessory device 102 may include forming the slots in the tubular or elongated components of the system 100 and/or accessory device 102 using these or other manufacturing steps.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example configuration being used in other configurations. The invention's scope is, of course, defined in the language in which the appended claims are expressed.

What is claimed is:

1. A steerable medical device comprising:
a proximal elongate shaft having a first plurality of lumens;
a distal elongate shaft having a second plurality of lumens, wherein the second plurality is less than the first plurality;
an articulation assembly coupling the proximal and distal elongate shafts, the articulation assembly comprising:
a segmented sleeve coupled to the proximal elongate shaft;
an articulating connector pivotably coupled to the segmented sleeve and coupled to the distal elongate shaft; and
at least one deflection member extending through a dedicated lumen of the proximal elongate shaft and coupled to the articulating connector;
wherein selective actuation of the at least one deflection member causes lateral deflection of the distal elongate shaft relative to the proximal elongate shaft.

2. The device of claim 1, further comprising an intermediate elongate shaft configured to connect corresponding lumens between the proximal and distal elongate shafts through the articulation assembly.

3. The device of claim 1, wherein the segmented sleeve comprises a first slot and a second slot extending from a proximal end thereof and a first aperture and a second aperture positioned between the first slot and the second slot.

4. The device of claim 3, wherein the articulating connector comprises a first linkage member and a second linkage member pivotably coupled to the segmented sleeve via pins extending through the first aperture and the second aperture.

5. The device of claim 1, wherein the at least one deflection member comprises a first deflection member and a second deflection member positioned on opposing sides of the articulating connector.

6. The device of claim 1, wherein the first plurality of lumens comprises five lumens and the second plurality of lumens comprises three lumens.

7. The device of claim 1, wherein the articulation assembly enables lateral deflection of the distal elongate shaft beyond 90° relative to a longitudinal axis of the proximal elongate shaft.

8. A medical device comprising:
an elongate shaft having a proximal elongate shaft and a distal elongate shaft;
the proximal elongate shaft comprising a first lumen configuration;
the distal elongate shaft comprising a second lumen configuration different from the first lumen configuration;
a hinge assembly coupling the proximal and distal sections, the hinge assembly comprising a clevis structure mounted on pivot pins; and
a first control element and a second control element each coupled to the clevis structure for controlling lateral deflection;
wherein actuation of the deflection members causes controlled lateral movement of the distal elongate shaft relative to the proximal elongate shaft.

9. The device of claim 8, wherein the first lumen configuration comprises a guidewire lumen, a contrast lumen, a steering wire lumen, and two or more control element lumens.

10. The device of claim 8, wherein the second lumen configuration comprises a guidewire lumen, a contrast lumen, and a wire lumen.

11. The device of claim 8, further comprising an intermediate elongate shaft defining a plurality of lumens, the plurality of lumens extending between corresponding lumens of the proximal and distal elongate shafts through the hinge assembly.

12. The device of claim 8, wherein the first control element and the second control element comprise deflection wires extending through dedicated lumens in the proximal section.

13. The device of claim 11, wherein the hinge assembly and the intermediate elongate shaft enable lateral deflection of the distal elongate shaft while maintaining lumen continuity between the proximal and distal elongate shafts.

14. The device of claim 8, wherein actuation of the control elements enables lateral deflection of the distal section beyond 90° relative to a longitudinal axis of the proximal section.

15. A medical system comprising:
a handle assembly;
a proximal elongate shaft extending from the handle assembly; an articulation assembly at a distal portion of the proximal elongate shaft;
a plurality of deflection members extending from the handle assembly through the proximal elongate shaft to the articulation assembly;
a distal elongate shaft extending distally from the articulation assembly;
wherein the handle assembly comprises actuation mechanisms configured to selectively tension the deflection members to control lateral movement of the distal elongate shaft.

16. The system of claim 15, wherein the handle assembly comprises a first actuator for controlling a cutting wire and a second actuator for controlling lateral deflection.

17. The system of claim 15, wherein the articulation assembly comprises a pivotable connector.

18. The system of claim 15, wherein the proximal elongate shaft comprises a first number of lumens and the distal elongate shaft comprises a second number of lumens less than the first number.

19. The system of claim 15, wherein the deflection members extend through dedicated lumens in the proximal elongate shaft.

20. The system of claim 15, wherein the articulation assembly enables controlled lateral deflection through at least 120° of movement.
